(19) United States
(12) Patent Application Publication
(10) Pub. No.: US 2019/0192560 A1
(43) Pub. Date: Jun. 27, 2019

(54) POLYMER MATERIAL COMPRISING AT LEAST ONE DIFFERENT DOPING ELEMENT, USES AND PRODUCTION METHOD

(71) Applicant: S-TECHS GmbH, Hamburg (DE)

(72) Inventor: Edgar-Johannes VAN HATTUM, Bergen (DE)

(21) Appl. No.: 16/326,947

(22) PCT Filed: Aug. 22, 2016
(86) PCT No.: PCT/EP2016/069816

§ 371 Date: Feb. 21, 2019

(51) Int. Cl. A61K 33/26; C08K 3/22; C08J 3/21; C09K 11/60; C09K 11/02; A61K 47/34; A61P 7/00; A43B 17/14; D01D 1/06; D01D 5/00; D01F 1/10; D01F 6/60; D01F 6/62; B29C 48/08; B29C 48/00; B29C 48/05; B29C 48/14; B29C 48/295

(52) U.S. Cl. CPC A61K 33/26; B29L 2031/507; C08J 3/212; C09K 11/602; C09K 11/02; A61K 47/34; A61P 7/00; A43B 17/14; D01D 1/065; D01D 5/003; D01F 1/106; D01F 6/605; D01F 6/62; B29C 48/08; B29C 48/022; B29C 48/05; B29C 48/142; B29C 48/295; C08K 2003/2265; C08K 2201/011; C08K 2201/005; D10B 2331/042; D10B 2331/021; C08K 3/22

(57) ABSTRACT

A polymer material comprises one or more different doping elements. The or at least one of the different doping elements at least partially absorbs an electromagnetic radiation emitted by a human or animal body and at least partially emits an electromagnetic radiation in an infrared range, preferably in an infrared C range. A textile material comprises the polymer material according to the invention. The invention further relates to medical and non-medical uses of the polymer material according to the invention and to a manufacturing method of the polymer material according to the invention.

### TECHNICAL FIELD OF THE INVENTION

The invention relates to a polymer material, comprising one or more different doping elements, in that the or at least one of the different doping elements at least partially absorbs an electromagnetic radiation emitted by the human or animal body, and at least partially emits an electromagnetic radiation in the infrared range, in particular in the infrared C range, and to a textile material comprising the polymer material according to the invention. The invention further relates to medical and non-medical applications of the polymer material according to the invention, and to a manufacturing method of the polymer material according to the invention.

### BACKGROUND OF THE INVENTION

Metabolically adequate transport methods occurring in the tissue by an exchange of substances between the blood and the tissue cells are a precondition for being able to obtain or restore physiological organ functions. Physiological organ functions are in turn a precondition for an adequate physical and mental efficiency of a human or animal organism. An important function is attached to the microcirculation of the blood, i.e. the perfusion states in the capillaries, arterioles and venules, as the transport phenomena of the exchange of substances with the tissue cells, i.e. the tissue nutrition, and the first steps of the immune response take place here (Klopp et al., “Änderung des Funktionszustandes der Mikrozirkulation durch eine adjuvante BioKorrektur-Behandlung bei Patienten mit Diabetes Mellitus Typ II”, Archiv Euromedica, 2014, Vol. 4, No. 1, page 36).

A circulatory regulation is optimally adapted to the changing metabolic requirements of the organs if it permits sufficiently fast flow velocities and a metabolically adequate distribution of the plasma-blood cell mixture in the microvascular network. The focus is here the endothelium-imparted, shear stress-dependent tone regulation of the large caliber and small caliber arterioles and their vasomotor activities related thereto. The main criteria for the functional state are thus the flow velocities, the flow and the distribution state of the plasma-blood cell mixture in the microvessels which are determined by the segregation phenomena of the plasma and the blood cells. Not only the mechanisms of temperature regulation, but particularly the tissue nutrition and the immune defense are thus arranged as needed. The respective oxygen extraction of the blood is an expression of the functional state. Therefore, the following applies: by influencing the microcirculation in a physiologically advantageous manner, this serves an optimum cell and organ function and an efficient immune defense produced naturally in the body.

Disorders or deficient functional states of the microcirculation impair organ function and in the end lead to organic damages and to an increased susceptibility to infection, wound healing disorders, ulcerations, etc. If previous damages are already present, e.g. in diabetic microangiopathy or in so-called peripheral circulatory disorders, organic damages in most cases first manifest themselves at the so-called predestination sites for tissue damages. This in particular concerns near-surface body regions with small radii of curvature, for example toes, ankles, etc. It should also be borne in mind that restrictions on microperfusion are at least also involved if stress and overstress damages prematurely occur in the bone-ligament-articulation system, such as fatigue-induced fractures.

It results therefrom that any measure leading to an improvement of the functional state of the microcirculation of the blood is effective in a prophylactic and adjuvant manner.

One example for the improvement of the functional state of the microcirculation is the adjuvant motion treatment called “BioCorrection”. In a placebo-controlled test series, it has been discovered that the motion treatment called “BioCorrection” at a defined treadmill exercise for patients suffering from Diabetes mellitus Type II stimulates a metabolically adequate microcirculation of the blood (Klopp et al., ibid.) and is thus adjuvantly effective.

Exactly against the background that some patients have only a very limited ability to move due to their disease and therefore their physical constitution, there is a need to provide an alternative possibility to stimulate the microcirculation of the blood in a metabolically adequate manner.

### SUMMARY OF THE INVENTION

The aforementioned needs are achieved by means of the objects as claimed according to the invention. Advantageous configurations are illustrated in the dependent claims and in the following specification.

Accordingly, a first object of invention of the present invention relates to a polymer material comprising one or more different doping elements, characterized in that the or at least one of the different doping elements at least partially absorbs an electromagnetic radiation emitted by the vertebrate, preferably the human being, and at least partially emits an electromagnetic radiation in the infrared range.

A second object of invention of the present invention relates to a textile material comprising a polymer material according to the invention.

A third object of invention of the present invention relates to a polymer material according to the invention for use as an adjuvant or as a prophylactic in therapeutic methods.

A fourth object of invention of the present invention relates to a polymer material according to the invention for use in the prophylactic or adjuvant treatment of a metabolically inadequate microcirculation of blood in the vertebrate, preferably the human being; in the treatment of necrotic vessels; in wound healing, preferably in the wound treatment for decubitus, diabetic foot syndrome, ulcus cruris, burns and for the wound treatment of many other chronic and secondarily healing wounds; of diabetes mellitus, in particular diabetes mellitus type I and/or diabetes mellitus type II; cancers; protein-related diseases such as Alzheimer's disease or dementia; thrombocyte diseases; erythrocyte diseases; immunological diseases such as immunological hyperactivity; infectious diseases such as wound infections; neurological diseases, in particular insofar as they are based on a disease of the sheath of the nerve cells and/or synapses.

A fifth object of invention of the present invention relates to a use of a polymer material according to the invention for the non-medical, in particular the athletic performance enhancement of a vertebrate, preferably a human being.

A sixth object of invention of the present invention relates to a method for the manufacture of a polymer material according to the invention, characterized in that the method comprises or consists of the following steps:
a. providing a suitable dissolved polymer,
b. providing a suitable doping element,
c. evaporating the doping element provided in step b) using appropriate methods, and incorporating the evaporated doping element into the dissolved polymer provided in step a), and
d. extruding the polymer material according to the invention in the electric field.

The object of invention described above may, if this is reasonable from the point of view of a skilled person, comprise any possible combination of the preferred configurations according to the invention which are disclosed below and, in particular, also in the dependent claims.

### DESCRIPTION OF THE FIGURES

FIG. 1 shows a diagram comprising measurement data as to the feature mean flow of red blood cells in the microvascular networks of the skeletal muscular system Q_{RBC} for control and verum.

FIG. 2 shows a diagram comprising measurement data as to the feature number of blood cell-perfused nodal points in the microvessel network nNP for control and verum.

FIG. 3 shows a diagram comprising measurement data as to the feature venule-side oxygen extraction in the microvascular networks of the skeletal muscular system ΔPO₂ for control and verum.

FIG. 4 shows a bar chart comprising differences in feature changes for venule-side oxygen extraction ΔPO₂ in the microvascular networks of the skeletal muscular system of the initial values on the 0th day to the measured values on the 30th day for control and verum.

FIGS. 5A and 5B show vital-microscopic example of findings of an elderly diabetic (subsample D), the verum group, before the examination (FIG. 5A) and after application of the insole according to the invention on the 30th day of the examination (5B).

### DETAILLED DESCRIPTION OF THE INVENTION

The present invention is based on the surprising findings that the polymer material according to the invention stimulates the microcirculation of the blood in a metabolically adequate manner upon body contact with the vertebrate, preferably the human being, and thus improves cell and tissue functions and the immune defense.

According to the present state of knowledge, it is assumed that the polymer material according to the invention comprising the one or more doping elements is excited by the radiation emitted by the vertebrate, preferably the human being, such that a material movement of the doping elements occurs in the polymer material according to the invention. The resulting kinetic energy generates a so-called carrier wave of the polymer material according to the invention.

At the same time, the radiation emitted by the vertebrate, preferably the human being, stimulates the polymer material according to the invention such that electromagnetic radiation in the infrared range, preferably in the infrared C range, more preferably in the wavelength range from 3 μm to 15 μm, more preferably in the range from 4 to 12 μm is at least partially emitted by the polymer material according to the invention.

It is currently assumed that the kinetic energy of the generated carrier wave modulates the simultaneously emitted electromagnetic radiation in the infrared range such that the infrared radiation can penetrate deeper into the tissue of the vertebrate, preferably the human being, and thus has a beneficial effect on microcirculation in the peripheral tissue. Here, the frequency and amplitude modulation correlate in such a way that a carrier wave in the range from more than 60 decibels (Db), preferably 80 to 13β Db, more preferably 86 to 120 Db the frequency of the infrared radiation emitted by the polymer material according to the invention (hereinafter also referred to as infrared wave), in particular infrared C radiation, so that the amplitude of the infrared wave increases. In other words, the infrared wave is accelerated by the carrier wave to accordingly penetrate deeper into the tissue of the vertebrate, preferably the human being.

In order that the polymer material according to the invention absorbs a sufficient amount of electromagnetic radiation emitted by the body of the vertebrate, preferably the human being, and that the infrared wave emitted by the polymer according to the invention can penetrate correspondingly deep into the tissue of the vertebrate, preferably the human being, it should be placed at an appropriate distance close to the body. Preferably, the polymer according to the invention is arranged at a distance of 0 to 5 cm from the skin of the vertebrate, preferably the human being, more preferably of up to 3 cm, even more preferably of up to 2 cm, even more preferably of up to 1 cm. If a covering material is placed between the body and the polymer material according to the invention in the textile material according to the invention, such as an insole, it may be necessary to break it through, for example to perforate it such that the radiation emitted by the body of the vertebrate, preferably the human being, can be absorbed by the polymer material according to the invention and the infrared wave emitted by the material according to the invention can penetrate the body in a suitable way.

In this respect, the present invention differs significantly from conventional irradiation of a vertebrate, in particular a human being, using infrared heat lamps, since this emitted infrared radiation cannot penetrate so deeply into the tissue.

The advantageous effect of the polymer material according to the invention, in particular of a textile material according to the invention on the microcirculation of blood in the vertebrate, preferably in the human being, was demonstrated by means of a placebo-controlled study of insoles according to the invention vs. control insoles (cf. detailed description in example embodiment C below).

In this study, it could be proven that on the one hand the mean flow of red blood cells in the microvascular networks of the skeletal muscular system Q_{RBC} could be significantly improved for the insole according to the invention (verum) compared to the control insole. This means that a sufficient pressure gradient between arterioles and venules was achieved as a precondition for a needs-based flow of the plasma-blood cell mixture in the microcirculation. By improving the mean flow of red blood cells in the microvascular networks of the skeletal muscular system Q_{RBC}, the adaptation width of Q_{RBC} to changing metabolic needs of the tissue to be supplied is improved.

In addition, the number of blood cell-perfused nodal points in the microvascular network nNP for the insole according to the invention (verum) was significantly increased compared to the control insole. This means that an appropriate needs-based distribution of the plasma-blood cell mixture in the capillary networks is achieved. This reduces the diffusion pathways while simultaneously having high microcirculatory reserves for the adaptation of nNP to modified activity states of the organs to be supplied.

Furthermore, it could be shown in the study that the venule-side oxygen extraction in the microvascular networks of the skeletal muscular system ΔPO₂ was significantly improved for the insole according to the invention (verum) compared to the control insole. A high oxygen extraction ΔPO₂, in case of the present improved flow Q_{RB}, leads to an improved supply of substrates for cellular reactions, such as oxygen, nutrients, amino acids, proteins, pharmaceutical agents, plasmatic and cellular factors of the immune defense and the glucose metabolism, etc. and removal of metabolic products, such as CO, CO₂.

Due to an improved microcirculation according to the invention, an improved supply of the cells or the tissue region with oxygen, nutrients, amino acids, proteins and pharmaceutical agents, for example, is on the one hand achieved, which improves the cell and thus also the organ functions. Due to the improved cell and organ functions, the body consequently also becomes more efficient, which has a positive effect when coping with diseases and wounds, but also in case of physically demanding activities, in particular corresponding sports.

On the other hand, the improved transport of plasmatic and cellular factors of the immune defense can lead to an improved immune defense. This is particularly beneficial in the treatment of wounds to reduce the risk of infection.

The risk of infection can also be reduced by the modulated infrared radiation, preferably the infrared C radiation, especially in the range from 3 to 15 μm, emitted by the polymer material according to the invention, since this also has a germ-reducing effect. This effect is mainly due to the fact that the emitted modulated infrared radiation is opaque to the absorption spectrum of water, in other words is trout to the water absorption spectrum.

As already described above, it is currently assumed that the improved microcirculation due to the application of an polymer material according to the invention is probably due to the fact that the polymer material according to the invention is excited by the electromagnetic radiation emitted by the body such that kinetic energy is generated due to the material movement of the doping elements in the polymer material, which produces a so-called carrier wave, and that at the same time this carrier wave has an influence on the infrared radiation emitted by the polymer material according to the invention and modulates this radiation in particular in such a way that it can penetrate deeper into the tissues and leads to an optimization of the oxygen binding of the hemeproteins in the peripheral blood vessels, in particular the arterioles, capillaries and venules. Hemeproteins, such as hemoglobin, myoglobin and cytochromes, belong to the most important physiological iron-containing compounds, the hemeproteins, in the “non-oxygen-stressed” state, including iron(II) complexes, so-called iron porphyrin-containing prosthetic groups (DGE/÷GE/SGE/SVE, 2000; Elmadfa and Leitzmann, 1990; Yip, 2001).

The emitted infrared radiation, preferably the infrared C radiation, changes, in particular “stretches” the molecular arrangement of the iron(II) complex, so that it is present in a spherical form which is more reactive to an oxygenation to the oxygen-containing iron(III) complex. The oxidation bridges between the Fe2O3 are removed or reduced to such an extent that a corresponding conformational change of the iron(II) complex takes place.

A hemeprotein, in particular hemoglobin, which comprises so-called “stretched” iron(II) complexes, can bind between 4% and 18% more oxygen and thus provide more oxygen to the cells and tissue regions. This advantage is particularly demonstrated by the feature venule-side oxygen extraction in the microvascular networks of the skeletal muscular system ΔPO₂ in the placebo-controlled study and by the feature relative hemoglobin saturation rHb (cf. example embodiment C).

In addition, it is assumed that the protein complex ferritin, which functions as an iron store or as a transport form of iron(III) in the body, is stimulated by the infrared C radiation emitted according to the invention to release more iron(II). The increased iron(II) concentration leads to the fact that more iron(II) can be bound in the hemeproteins and thus to an increase of the oxygen transport into the cells and tissues, or thus permits a removal of CO₂ from the tissue.

The first object of invention thus relates to a polymer material comprising one or more different doping elements, characterized in that the or at least one of the different doping elements at least partially absorbs an electromagnetic radiation emitted by the vertebrate, preferably the human being, and at least partially emits an electromagnetic radiation in the infrared range.

Electromagnetic radiation in the infrared range includes the spectral range between 10⁻³ m and 7.8×10⁻⁷ m (1 mm and 780 nm), which corresponds to a frequency range from 3×10¹¹ Hz to approx. 4×10¹⁴ Hz (300 GHz to 400 THz). The infrared range itself is once again divided into the near infrared range (NIR), namely infrared A (780 nm to 1,400 nm) and infrared B (1,400 nm to 3,000 nm), the intermediate (MIR) and the far infrared range (FIR), namely infrared C (MIR: 3,000 nm to 50 μm, FIR: 50 μm to 1 mm). Preferably, the polymer material according to the invention emits in accordance with all configurations according to the invention electromagnetic radiation in the infrared C range, preferably in the range from 3 μm to 50 μm, more preferably in the range from 3 μm to 20 μm, alternatively of 4 μm to 15 μm, alternatively of 5 μm to 12 μm.

According to the invention, any polymer to be used for a textile material may be used as a carrier for the polymer material according to the invention of the first object of invention. Preferably, the polymer material according to the first object of invention is characterized in that the polymer is selected from the list consisting of the group of the polyesters, for example polyethylene terephthalate (PET), the group of the polyam ides, for example poly(p-phenylene terephthalamide) (PPTA), and poly(m-phenylene isophthalamide) (PMPI), and more preferably consists of polyethylene terephthalate (PET).

In a further cumulatively or alternatively preferred configuration, the polymer material according to the invention is characterized in that the or at least one of the different doping elements comprises an iron alloy, preferably an iron oxide alloy (ferrites), more preferably an alkaline earth-iron oxide alloy (alkaline earth ferrites), in particular barium or strontium ferrites, particularly preferably a SrFe₁₂O₁₉ alloy (SrO(γ Fe₂O₃)₆).

The following alloys are preferably excluded from protection from the present invention in all objects of invention: MnFe-phosphorus compounds, preferably MnFe(As,P_{w}GeₓSi_{z})ₛ, in particular wherein x=0.3-0.7 and/or w is less than or equal to 1−x and z=1−x−w, and/or FeMn-phosphorus compounds containing As, Si-phosphorus substitution and, if required, in combination with La(FeMnP)AlCo; alloy containing FeMnP_{0.7}Ge_{0.3}; alloy containing FeMnP_{0.5}Ge_{0.5}; alloy containing Fe_{0.86}Mn_{1.14}P_{0.5}Si_{0.35}Ge_{0.15}; and/or alloys containing MnZn.

In accordance with the invention, the doping elements are incorporated into the polymer carrier of the polymer material according to the invention in a sufficient quantity so that the emitted infrared wave positively influences the microcirculation. Preferably, the doping elements have to the polymer of the polymer material according to the invention a weight ratio of 1:9 to 9:1, more preferably of 2:8 to 8:1, more preferably of 3:7 to 7:3, even more preferably of 4:6 to 6:4, alternatively of 1:1. It is assumed here, that 1 L polymer solution corresponds to 1 kg. Preferably, the polymer material according to the invention comprises in terms of weight less doping element than polymer, <1:1, preferably 4:6 parts by weight.

In a further alternatively or cumulatively preferred configuration, the polymer material according to the invention is characterized in that the different doping element(s) is/are incorporated into the inside of the polymer material, and the polymer material isolates the doping elements with respect to the polymer material surface. In other words, in accordance with the invention, the doping element(s) is/are not arranged on the surface of the polymer material. This isolation with respect to the surface of the polymer material according to the invention can be achieved in particular by the E-spinning method to be used in accordance with the invention. Under tension and stress of the electric field, the initially externally arranged doping elements are removed from the surface of the polymer material according to the invention both by oxidation and mechanically by adhesion, or are very significantly reduced.

In a further alternatively or cumulatively preferred configuration, the polymer material according to the invention is characterized in that the doping element(s) is/are incorporated in an inhomogeneous manner into the polymer carrier. The inventor currently assumes that an inhomogeneous distribution of the doping element leads to a further improvement of the carrier wave or of the emitted infrared wave of the polymer material according to the invention.

In a further alternatively or cumulatively preferred configuration, the polymer material according to the invention is characterized in that the or at least one of the different doping elements has a size in the range from 1 to 10 nm and is provided with a spacing such that electron clouds of two doping elements overlap at least in regions. In other words, the polymer material according to the invention comprises the or at least one of the different doping elements at least in sections in such a distribution in the polymer carrier that due to this arrangement, in particular the overlapping in regions of the electron clouds of the different doping elements, a kinetic energy is produced which causes a carrier wave of the polymer material. This is based on electron migration and on a physical effect, the so-called “skin effect”, which has to be extended in the area of direct current and electron hopping. Every change of the direction of travel generates an impulse, the so-called “sound”. In case of a parallel connection, the voltages add up in contrast to a series connection or a serial electron run.

The alternatively or cumulatively preferred configurations of the first object of invention according to the invention can be realized in any technically meaningful combination. Features of the examples embodiments can be used individually or in combination with features of the detailed description if this is technically meaningful.

According to the second object of invention, a textile material comprising a polymer material according to the invention is claimed. The preferred configurations with respect to the features of the polymer material according to the in invention and in accordance with the first object of invention are also applicable to the present second object of invention of the textile material.

According to a preferred configuration, the textile material according to the invention is selected from the group consisting of clothing, preferably outerwear, underwear, stockings comprising surgical stockings, T-shirts, long-sleeved shirts, pants, in particular running pants, shoes, in particular shoe upper, inner lining and insole; mattress pad; duvet cover; pillow case; seat cover; and dressing material for wound care. The textile material according to the invention may comprise the polymer material according to the invention in all its possible configurations according to the invention, the polymer material being, for example, at least partially processed, in particular woven, knitted, warp or weft knitted or knotted as a polymer fiber or being integrated as a film, or the surface of a textile material being at least partially coated, in particular laminated, with the polymer material according to the invention.

According to the third object of invention, the polymer material according to the invention is claimed for use as an adjuvant or as a prophylactic in therapeutic methods. The object of the invention also includes all configurations relating to the features of the polymer material of the invention in accordance with the first object of the invention. Therefore, in accordance with the present invention, the first application of the corresponding doping elements in polymer materials, in particular iron alloys, preferably an iron oxide alloy (ferrites), more preferably an alkaline earth-iron oxide alloy (alkaline earth ferrites), in particular barium ferrites or strontium ferrites, particularly preferably a SrFe₁₂O₁₉ alloy (SrO(γ Fe₂O₃)₆) comprised in polymer materials, in therapeutic methods is taught.

According to the fourth object of invention, the application of the polymer material according to the invention in therapeutic methods is specialized, in particular in the prophylactic or adjuvant treatment of a metabolically inadequate microcirculation of blood in the vertebrate, preferably the human being; in the treatment of necrotic vessels; in wound healing, for example for decubitus, diabetic foot syndrome, ulcus cruris, burns and for the wound treatment of many other chronic and secondarily healing wounds; of diabetes mellitus, in particular diabetes mellitus type I and/or diabetes mellitus type II; cancers; protein-related diseases such as Alzheimer's disease or dementia; thrombocyte diseases; erythrocyte diseases; immunological diseases such as immunological hyperactivity; infectious diseases such as wound infections; neurological diseases, in particular insofar as they are based on a disease of the sheath of the nerve cells and/or synapses.

According to a fifth object of invention, the polymer material according to the invention is claimed for the non-medical, in particular the athletic performance enhancement of a vertebrate, preferably a human being. Due to the improved microcirculation of the blood and the resulting improved supply of the cells or the tissue region with oxygen, nutrients, amino acids and proteins, for example, the cell or organ functions are improved, and the physical and mental performance, in particular the athletic performance, is thus increased.

According to the sixth object of invention, a method for the manufacture of a polymer material according to the invention is provided, all preferred configurations of the first object of invention also applying to the method according to the invention. The method according to the invention is characterized in that it comprises or consists of the following steps:
a. providing a suitable dissolved polymer,
b. providing a suitable doping element,
c. evaporating the doping element provided in step b) using appropriate methods, and incorporating the evaporated doping element into the dissolved polymer provided in step a), and
d. extruding the polymer material according to the invention in the electric field.

The polymer to be used in accordance with the invention according to all configurations of the first object of invention is dissolved in suitable solvents so that the evaporated doping element can be introduced into the dissolved polymer.

The doping element to be used according to the invention in accordance with all configurations of the first object of invention is preferably introduced into the dissolved polymer such that the polymer isolates the doping elements in the extruded polymer material according to the invention with respect to the polymer material surface. The doping elements are preferably distributed in an inhomogeneous manner in the extruded polymer material according to the invention, the individual doping elements being present in a molecular size, preferably in the range from 1 to 10 nm.

According to an alternatively or cumulatively preferred configuration, the method according to the invention is characterized in that in step c), the doping element is evaporated by means of a suitable evaporation technology, preferably by plasma vapor deposition (abbreviated PVD), in particular by a thermal (vacuum) evaporation technique, sputtering or similar techniques, and is deposited in the solution of the polymer. Alternatively, the doping element can be transferred into the gas phase by magnetron evaporation, for example by using microwave radiation, and a polymer-coated plasma can thus be produced by suddenly heating the doping elements and the polymer in an enclosed space. In this method, the particle sizes however vary very significantly. The smaller the amount of polymer into which the evaporated doping elements are introduced, the less gravity and inertia phenomena influence the production of the polymer material according to the invention.

According to an alternatively or cumulatively preferred configuration, the method according to the invention is characterized in that in step d), the polymer material is extruded by means of a suitable extrusion technique, in particular by exploiting an electric field, such as the electro-spinning technology, in particular blow spinning.

According to an alternatively or cumulatively preferred configuration, the method according to the invention is characterized in that one or more of the method steps are carried out under sterile conditions and/or in a vacuum.

In a further alternatively or cumulatively preferred configuration, in the method according to the invention for the manufacture of a polymer material, the polymer material is extruded as a suitable fiber or film which can be used in the manufacture of textile materials according to the invention in accordance with the second object of invention. Alternatively or cumulatively, the polymer material according to the invention may be extruded so as to coat at least parts of the textile material, preferably those parts which are in (direct) contact with the body of a vertebrate, preferably a human being. The coating of textile carrier material is preferably used for textile materials from the field of wound care (wound dressings, plasters, surgical drapes, etc.).

The present invention is further described on the basis of exemplary types of embodiment which are to be understood only as examples and which are not intended to limit the scope of protection of the present property right to these embodiments. The individual features of the following example embodiments can preferably be used separately or in (partial) combinations.

### EXAMPLE EMBODIMENTS

A: Manufacture of a Polymer Material According to the Invention

The doping elements according to the invention, for example a SrFe₁₂O₁₉ alloy (SrO(γ Fe₂O₃)₆), are metal-pyrolytically evaporated, preferably by means of a plasma vacuum evaporation technology. The produced gas is incorporated by injection into a suitable amount of a dissolved polymer, preferably a polyester, in particular polyethylene terephthalate, or of a polyamide. By applying an electric field, a polymer material fiber according to the invention is for example spun. An E-spinning method is preferably used for this purpose, a wire being drawn through a dissolved drop of polymer in which the evaporated doping element is incorporated. The diameter and the length of the wire are determined by the distance of the magnet to the dissolved polymer and the strength of the electric field.

The weight ratio of the doping element to the polymer in the resulting polymer material according to the invention which is spun as fiber is 4:6.

B: Manufacture of a Textile Material According to the Invention and of a Control

B.1: Control Insole for the Placebo Control

For the double-blind placebo-controlled examination described below, a commercially available standard insole from ECCO was used, which does not contain a polymer material according to the invention as an absorber layer and whose upper was subsequently perforated such that its structure corresponds to the insole according to the invention.

B.2: Manufacture of an Insole According to the Invention

For the double-blind placebo-controlled examination described below, an insole according to the invention is used, the structure of which is comparable to the above-mentioned control insole, the absorber layer containing the polymer material according to the invention (cf. example A) and being covered by the perforated upper (cf. control insole according to B1).

C: Placebo-Controlled Examinations for Influencing the Microcirculation

C.1: Design of the Examinations

A total sample N_{ges}=72 test persons was examined, divided into 4 sufficiently homogeneous subsamples each n=18 (each including 9 male and 9 female test persons).

TABLE 1
Presentation of subsamples, test person or patient population
and age of the placebo-controlled examination:

| Subsample | Test person or patient population | Age |
|---|---|---|
| A | Healthy untrained test persons | ≈25 years |
| B | Healthy trained test persons | ≈25 years |
| C | Older rehabilitated persons | ≈54 years |
| | (physical conditioning) | |
| D | Older diabetics | ≈55 years |
| | (Diabetes mellitus Type II, controlled) | |

GCP-conform inclusion and exclusion criteria.

The examinations were carried out in a blinded manner. Each test person in each subsample participated in two test series: use of a placebo insole (control) and use of the insole according to the invention (verum). A time interval of 2 to 3 weeks lay between the two test series. The order in which each test person participated in the test series control or test series verum was determined by a random generator.

The examinations were carried out under defined physical activity of the test persons on a treadmill with defined speed, which corresponded to a slightly accelerated walk. The treadmill stress took place daily at a time interval of 60 minutes during the examination period of 30 days, with a treadmill inclination of 5%, a mean treadmill speed of 0.8 to 1.0 m/s, starting with a low treadmill speed and a stepwise increase of 0.1 to 0.2 m/s every 10 minutes during the treatment of 60 minutes.

A non-invasive high-resolution measuring method based on the latest state-of-the-art science and technology, the combined white light spectroscopy and LaserDOPPLER micro-flux measurement (System LEA, Germany) served as the examination method.

Measurements were taken in a representative target tissue simultaneously in two tissue depths: 2 mm and 8 mm. The subcutis and skeletal muscular system in the left calf were selected as the defined target tissue of the measurements. Measurements were taken on the respective treatment day immediately before the start of the stress (initial values), during the stress and immediately after the end of the 60-minute treadmill stress. The measured values are collected under constant boundary conditions, with comfortable seats under constant macrocirculatory and temperature-regulatory boundary conditions. No alcohol, no coffee, no tea or cola drink two hours before the examinations. At least 6 hours sleep daily, no biotropic weather conditions in the observation interval.

The following features of the functional state of the microcirculation were in particular determined:
Flow of the red blood cells in the microvessels Q_{RBC}
Mean flow velocities of red blood cells in the microvascular networks VRsc
Relative hemoglobin saturation in the microvessels rHb
Number of blood cell-perfused nodal points in the defined network nNP
Venule-side oxygen extraction in the microcirculation of the target tissue ΔPO₂
   Times of measurement:

On day 0, the initial values were measured, on days 1 to 30, measurements were taken daily before and after treadmill stress.

A parameter-free test method with high selectivity, the WILCOXON rank sum test at the significance level alpha=5% was used for statistical evaluation of the measurement data obtained.

C.2: Examination Results

Of outstanding importance for a medical evaluation of the therapeutic success of the tested special insole on the functional state of the microcirculation are the features mean flow of the red blood cells in the microvessels Q_{RBC}, number of blood cell-perfused nodal points in the microvessel network nNP, and venule-side oxygen extraction ΔPO₂ in the active muscle tissue.

C.2.1: Feature Mean Flow of the Red Blood Cells in the Microvessels Q_{RBC}

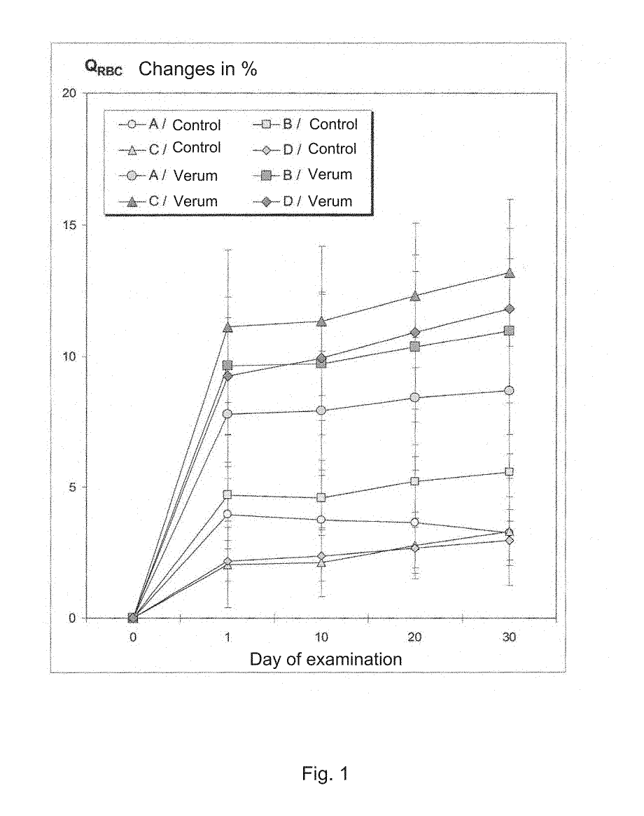

FIG. 1 shows the measurement data as to the feature mean flow of the red blood cells in the microvascular networks of the skeletal muscular system Q_{RBC} in the left calf of the test person/patient (mean values and standard deviations) in the subsamples A, B, C and D for control and verum (mean values and standard deviations). Ordinate: Changes in feature in percent (compared to the initial values). Abscissa: days of measurement in the 30-day examination period.

Depending on the age and the physical constitution of the test persons or patients, different changes in feature occur. The feature changes in the placebo groups (controls) reach a maximum of −5% on the 0th day compared to their respective initial values at time t=0. After the use of the insole according to the invention (verum groups), significantly higher amounts of the feature changes occur, some of which reach values that are more than twice as high.

C.2.2: Feature Number of the Blood Cell-Perfused Nodal Points in the Microvessel Network nNP

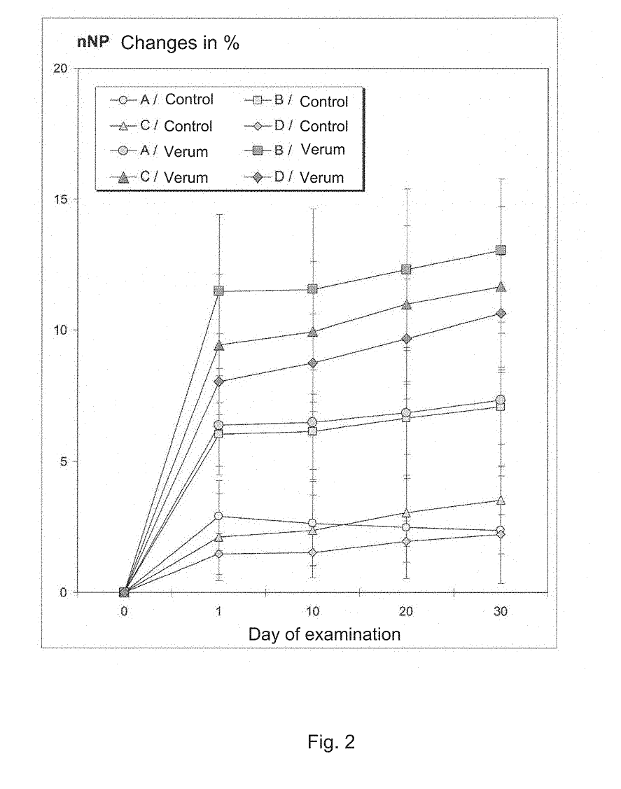

FIG. 2 shows the measurement data as to the feature number of the blood cell-perfused nodal points in the microvessel network nNP in the left calf of the test person/patient (mean values and standard deviations) for the subsamples A, B, C and D for control and verum (mean values and standard deviations). Ordinate: Changes in feature in percent (compared to the initial values). Abscissa: days of measurement in the 30-day examination period.

The feature changes as to nNP show a corresponding behavior to the feature changes of Q_{RBC}. In other words: Depending on the age and the physical constitution of the test persons or patients, different feature changes occur, wherein also with this feature significantly higher amounts of the feature changes occur after the use of the insole according to the invention (verum group), which in part reach values that are more than twice as high.

C.2.3: Feature Venule-Side Oxygen Extraction ΔPO₂

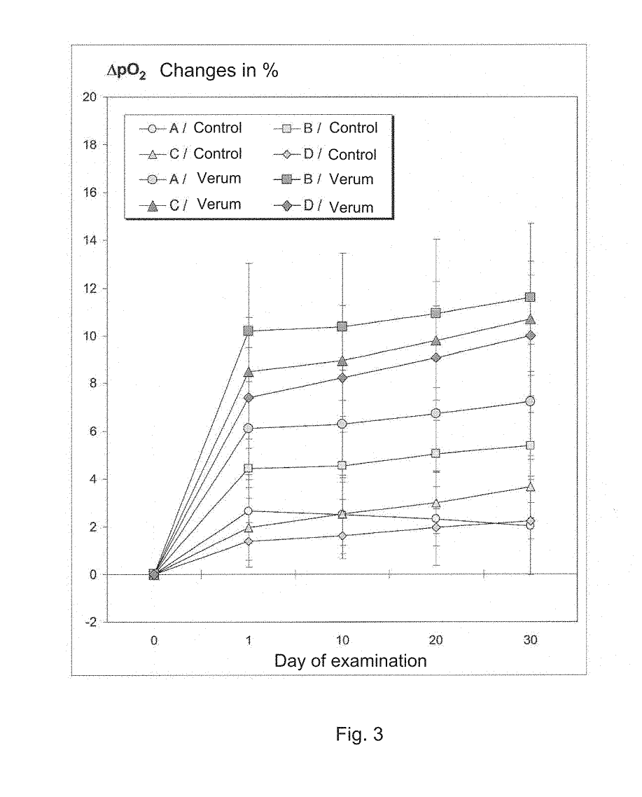

FIG. 3 shows the measurement data as to the feature venule-side oxygen extraction in the microvascular networks of the skeletal muscular system ΔPO₂ in the left calf of the test person/patient (mean values and standard deviations) for the subsamples A, B, C and D for control and verum (mean values and standard deviations). Ordinate: Changes in feature in percent (compared to the initial values). Abscissa: days of measurement in the 30-day examination period.

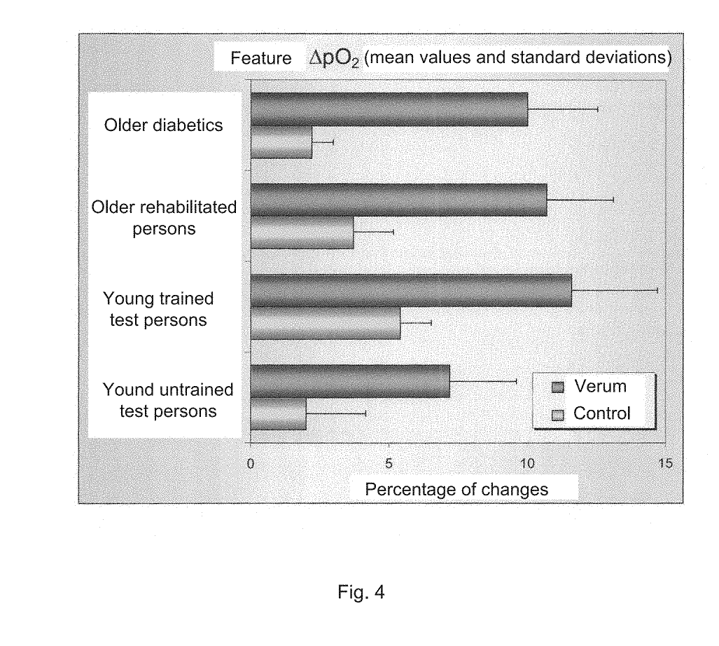

FIG. 4 shows the differences of the feature changes as to the venule-side oxygen extraction ΔPO₂ in the microvascular networks of the skeletal muscular system of the left calf of the test person/patient (mean values and standard deviations) of the initial values on the 0th day to the measured values on the 30th day in the subsamples A, B, C and D for control and verum (mean values and standard deviations) as a bar chart.

The feature changes as to APO2show a corresponding behavior to the feature changes of Q_{RBC} and/or ΔPO₂. In other words: Depending on the age and the physical constitution of the test persons or patients, different feature changes occur, wherein also with this feature significantly higher amounts of the feature changes occur after the use of the insole according to the invention (verum group), which in particular reach values between 2 and 5 times as high.

A therapeutic success of the insole according to the invention is evident for both younger and older test persons and patients. The largest differences between control (placebo) and verum (insole according to the invention) were found in older diabetics and older rehabilitated persons.

TABLE 2
Percentage of changes of the venule-side oxygen extraction
ΔPO₂ in the skeletal muscular system of the left calf
on the 30 th day compared to the respective initial values
on the 0 th day (mean values and standard deviations):

| | Control | Insole according to |
|---|---|---|
| Subsamples | (Placebo) | the invention (verum) |
| A: healthy untrained test persons | 2.0 (2.16) | 7.2 (2.41) |
| (age approx. 25 years) | | |
| B: healthy trained test persons | 5.4 (1.13) | 11.6 (3.11) |
| (age approx. 25 years) | | |
| C: older rehabilitated persons | 3.7 (1.46) | 10.7 (2.41) |
| (age approx.54 years) | | |
| D: older diabetics | 2.2 (0.77) | 10.0 (2.54) |
| (age approx. 55 year) | | |

It can furthermore be deduced from the examination results according to FIGS. 1 to 4 that there is a functional relationship between the behavior of the features Q_{RBC} and nNP and the feature venule-side oxygen extraction ΔPO₂.

C.2.4: Vital-Microscopic Examples of Findings From an Older Diabetic

(D), Verum (Insole According to Invention)

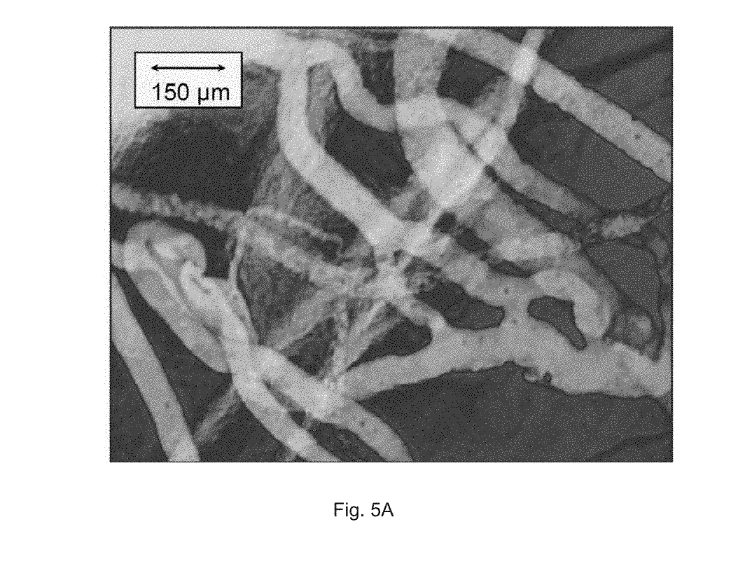

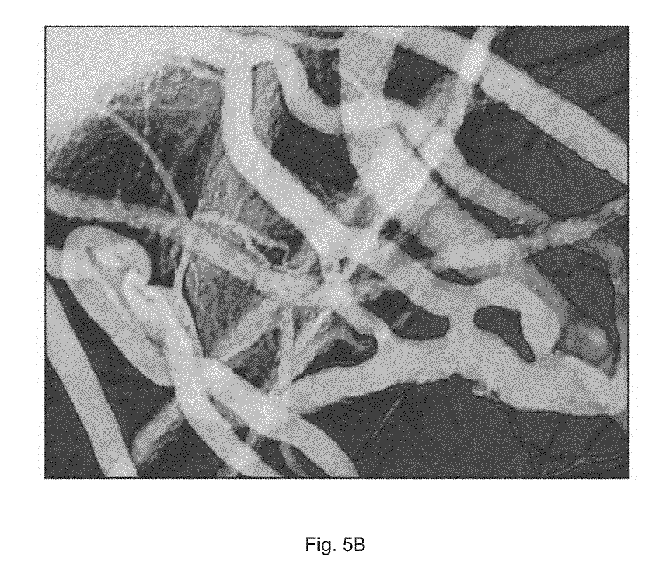

FIGS. 5A and 5B show vital-microscopic examples of findings of an older diabetic (subsample D) of the verum group, before examination (FIG. 5A) and after application of the insole according to the invention on the 30th day of the examination (5B). An area of the subcutis of the left calf (capillaries, arterioles, venules) is shown.

In FIGS. 5A and 5B, the blood cell-perfused microvessels are marked YELLOW by a pseudo-color transformation of the primary image, which corresponds to a bright gray in a black-and-white representation.

The distribution state of the plasma-blood cell mixture in the same region of the tissue is shown at two different observation times:

Before (base condition on the 0th day)

Afterwards (distribution state on the 30th day after the use of the insole according to the invention)

The representations of FIGS. 5A and 5B show the clear increase in blood cell-perfused microvessels and thus an extension of the microcirculatory reserve.

C.2.5: Mean Flow Velocities of the Red Blood Cells in the Microvessel Networks V_{Rsc} and Relative Hemoglobin Saturation in the Microvessels rHb

The measurement data as to the feature mean flow velocities of the red blood cells in the microvascular network of the skeletal muscular system V_{Rsc} in the left calf of the test person/patient in the subsamples A, B, C and D for control and verum surprisingly show that the red blood cells flow by a factor of up to 1.4 faster than the plasma.

The measurement data as to the feature relative hemoglobin saturation in the microvessels rHb in the left calf of the test person/patient in the subsamples A, B, C and D for control and verum surprisingly show a short-term doubling of the relative hemoglobin saturation rHb.

In other words, the red blood cells in the verum group are faster and redder than the red blood cells in the control group.

1. A polymer material comprising:
one or more different doping elements, wherein the or at least one of the different doping elements at least partially absorbs an electromagnetic radiation emitted by a vertebrate, and at least partially emits an electromagnetic radiation in an infrared range.

2. The polymer material according to claim 1, wherein the polymer material is selected from a list that includes a group of polyesters, for example polyethylene terephthalate (PET), a group of the polyamides, for example poly(p-phenylene terephthalamide) (PPTA) and Poly(m-phenylene isophthalamide) (PMPI).

3. The polymer material according to claim 1, wherein the or at least one of the different doping elements comprises an iron alloy.

4. The polymer material according to claim 1, wherein the or the different doping elements are incorporated into an inside of the polymer material and in that the polymer material isolates the different doping elements with respect to a polymer material surface.

5. The polymer material according to claim 1, wherein the or at least one of the different doping elements has a size in a range from 1 to 10 nm and is provided with a spacing such that electron clouds of two doping elements overlap at least in regions.

6. The polymer material according to claims 1, wherein the emitted infrared radiation has a wavelength in a range from 3 to 15 μm.

7. A textile material comprising a polymer material according to claim 1.

8. The textile material according to claim 7, whenein the textile material is selected from a group that includes clothing, preferably outerwear, underwear, stockings comprising surgical stockings. T-shirts, long-sleeved shirts, pants, in particular running pants, shoes, in particular shoe upper, inner lining and insole; mattress pad; duvet cover; pillow case; seat cover; and dressing material for wound care.

9. The polymer material according to claim 1 configured for use as an adjuvant or as a prophylactic in therapeutic methods.

10. The polymer material according to claim 1 configured for use in a prophylactic or adjuvant treatment of a metabolically inadequate microcirculation of blood in the vertebrate; in a treatment of necrotic vessels; in wound healing; of diabetes mellitus; cancers; protein-related diseases; thrombocyte diseases; erythrocyte diseases; immunologic diseases; infectious diseases; neurologic diseases, in particular insofar as they are based on a disease of the sheath of the nerve cells and/or synapses.

11. A use of the polymer material according to claim 1 for a non-medical, in particular athletic performance, enhancement of the vertebrate.

12. A method of manufacturing the polymer material according to claim 1, wherein the method comprises or consists of the following steps:
a. providing a suitable dissolved polymer,
b. providing a suitable doping clement,
c. evaporating the suitable doping element provided in step b) using appropriate methods, and incorporating an evaporated doping element into the suitable dissolved polymer provided in step a), and
d. extruding the polymer material according to claim 1 in an electric field.

13. The method of manufacturing the polymer material according to claim 12, wherein in step c), the suitable doping element is evaporated by a plasma evaporation technology (PVD) and is deposited into a solution of the polymer material, and/or in that in step d), the polymer material is extruded by an electrospinning technology, and/or in that one or more of the method steps are carried out under sterile conditions and/or in a vacuum.

14. The method of manufacturing a polymer material according to claim 12, wherein the suitable doping elements are embedded into the polymer material with a size of 1 to 10 nm and in that the polymer material isolates the suitable doping elements with respect to a polymer material surface.

15. The method of manufacturing a polymer material according to claim 12, wherein the polymer material is extruded as a fiber or as a foil, or in that further textile materials are at least partially coated with the polymer material.

16. The polymer material according to claim 1, wherein the vertebrate is a human being.

17. The polymer material according to claim 3, wherein the iron ally comprises an iron oxide alloy.

18. The polymer material according to claim 17, wherein the iron oxide alloy comprises an alkaline earth-iron oxide alloy.

19. The polymer material according to claim 18, wherein the alkaline earth-iron oxide alloy comprises a SrFe12O19 alloy.
